(19) United States
(12) Patent Application Publication
(10) Pub. No.: US 2021/0145908 A1
(43) Pub. Date: May 20, 2021

(54) COMPOSITIONS COMPRISING FUCOXANTHIN AND USE THEREOF IN PREVENTION AND TREATMENT OF FATTY LIVER DISEASE

(71) Applicant: ALGATECHNOLOGIES LTD., Kibbutz Ketura (IL)

(72) Inventors: Tair LAPIDOT, Lapid (IL); Eyal AFERGAN, Kiryat Ata (IL); Yael CARRASSO, Kibbutz Yotvata (IL); Hagai STADLER, Ra'anana (IL)

(21) Appl. No.: 17/045,171

(22) PCT Filed: Apr. 4, 2019
(86) PCT No.: PCT/IL2019/050390

§ 371 Date: Oct. 4, 2020

Related U.S. Application Data
(60) Provisional application No. 62/652,401, filed on Apr. 4, 2018.

(51) Int. Cl. A61K 36/03; A61K 47/44; A61K 31/336; A61P 1/16; A61P 13/12

(52) U.S. Cl. CPC A61K 36/03; A61K 47/44; A61P 13/12; A61P 1/16; A61K 31/336

(57) ABSTRACT

The present invention is directed to compositions comprising microalgae extracts comprising fucoxanthin. Further provided are compositions and methods for reducing lipid accumulation in cells. Also provided are methods for prevention or treatment of metabolic syndrome, including but not limited to a fatty liver disease, or a fatty kidney disease.

### CROSS-REFERENCE TO RELATED APPLICATIONS

This application claims the benefit of priority of U.S. Provisional Patent Application No. 62/652,401 titled “COMPOSITIONS COMPRISING FUCOXANTHIN AND USE THEREOF IN PREVENTION AND TREATMENT OF FATTY LIVER DISEASE”, filed Apr. 4, 2018, the contents of which is incorporated herein by reference in its entirety.

### FIELD OF INVENTION

The present invention is directed to, inter alia, compositions comprising fucoxanthin or metabolites thereof, and use thereof in reduction of fat accumulation in cells or the prevention and treatment of metabolic diseases.

### BACKGROUND OF THE INVENTION

Fatty liver disease (FLD) is a prevalent liver condition that occurs when lipids accumulate in liver cells. The lipid accumulation causes cellular injury and sensitizes the liver to further injuries. The accumulated lipids may also impair hepatic microvascular circulation.

FLD may arise from a number of sources, including excessive alcohol consumption and metabolic disorders, such as those associated with insulin resistance, obesity, and hypertension. Nonalcoholic fatty liver disease (NAFLD) may also result from metabolic disorders such as, e.g., galactosemia, glycogen storage diseases, homocystinuria, and tyrosemia, as well as dietary conditions such as malnutrition, total parenteral nutrition, starvation, and overnutrition. In certain cases, NAFLD is associated with jejunal bypass surgery. Other causes include exposure to certain chemicals such as, e.g., hydrocarbon solvents, and certain medications, such as, e.g., amiodarone, corticosteroids, estrogens (e.g., synthetic estrogens), tamoxifen, maleate, methotrexate, and nucleoside analogs. Acute fatty liver conditions can also arise during pregnancy.

FLD can progress to more advanced liver disease such as nonalcoholic steatohepatitis (NASH; metabolic steatohepatitis), a condition characterized by liver inflammation and damage, often accompanied by fibrosis or cirrhosis of the liver. NASH may progress to further liver damage ultimately leading to chronic liver failure and, in some cases, hepatocellular carcinoma.

### SUMMARY OF THE INVENTION

The present invention provides compositions comprising fucoxanthin and/or metabolites thereof, and methods of use thereof for reduction of lipid content in cells, e.g., hepatocytes, and kidney cells. Further provided are methods for prevention or treatment of metabolic diseases, such as fatty liver disease, or fatty kidney disease.

According to one aspect, there is provided a method for reducing lipid content in a cell, the method comprising the step of contacting the cell with a composition comprising microalgae extract and a carrier, thereby decreasing lipid accumulation in the cell, thereby reducing the lipid content in the cell.

According to another aspect, there is provided a method of preventing or treating a metabolic syndrome in a subject in need thereof, the method comprising administering to the subject a therapeutically effective amount of a composition comprising microalgae extract and a carrier, thereby preventing or treating a metabolic syndrome in the subject.

In some embodiments, the cell is an ectopic fat accumulating cell.

In some embodiments, the ectopic fat accumulating cell is selected from the group consisting of: a liver cell, a kidney cell, a pancreatic cell, and a muscle cell.

In some embodiments, the metabolic syndrome is selected from the group consisting of: a fatty liver disease, and a fatty kidney disease.

In some embodiments, the microalgae extract is a Phaeodactylum extract.

In some embodiments, the Phaeodactylum extract comprises at least 0.5% (w/w) fucoxanthin by dry weight.

In some embodiments, Phaeodactylum is Phaeodactylum tricornutum.

In some embodiments, the carrier is a plant-derived oil.

In some embodiments, the plant-derived oil comprises at least 85% fatty acid chains comprising 16 carbon atoms at most.

In some embodiments, the plant-derived oil comprises at least 85% saturated fatty acid chains.

In some embodiments, the plant-derived oil is coconut oil or medium-chain triglyceride (MCT) oil.

In some embodiments, the microalgae extract is a fraction of a microalgae extract.

In some embodiments, the fraction comprises at least 0.03 μg/mL fucoxanthin.

In some embodiments, the fraction comprises one or more compounds selected from the group consisting of: palmitic acid, arachidonic acid, eicosapentaenic acid, and docosahexaenoic acid.

In some embodiments, the method if for preventing or treating fat-induced hepatotoxicity, nephrotoxicity, or both.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a bar graph depicting the effect of various concentrations (μg/mL) of the fucoxanthin composition of the invention on lipid accumulation inhibition, under a fatty liver model of Oleate:Palmitate (O:P; 0.3 mM) in human hepatocytes (Hep G2), compared to control (“Veh”). * p<0.0001 vs. Vehicle control without O:P; #p<0.0001 vs. Vehicle control with O:P; $ p<0.0099 vs. Vehicle control with O:P; and \hat{ } p<0.0009 vs. Vehicle control with O:P.

FIG. 2 is a bar graph depicting the effect of the fucoxanthin composition of the invention (440 μg/mL) on lipid accumulation inhibition, under a fatty liver model of Oleate:Palmitate (O:P; 0.3 mM) in human hepatocytes (Hep G2), compared to control (“Veh”), and to Docosahexaenoic acid (DHA) at various concentrations (presented as μM and μg/mL). * p<0.0001 vs. Vehicle control without O:P; and #p<0.0001 vs. Vehicle control with O:P.

FIG. 3 is a bar graph depicting the effect of the fucoxanthin composition of the invention (440 μg/mL) on lipid accumulation inhibition, under a fatty liver model of Oleate:Palmitate (O:P; 0.3 mM) in human hepatocytes (Hep G2), compared to control (“Veh”), and to Eicosapentaenoic acid (EPA) at various concentrations (presented as μM and μg/mL). * p<0.0001 vs. Vehicle control without O:P; and #p<0.0001 vs. Vehicle control with O:P.

FIG. 4 is a bar graph depicting the effect of the fucoxanthin composition of the invention (440 μg/mL) on lipid accumulation inhibition, under a fatty liver model of Oleate:Palmitate (O:P; 0.3 mM) in human hepatocytes (Hep G2), compared to control (“Veh”), and to commercial fucoxanthin at various concentration (presented as μM and μg/mL). * p<0.0003 vs. Vehicle to control without O:P; #p<0.0001 vs. Vehicle control with O:P; and $ p<0.0004 vs. Vehicle control with 0:P.

FIG. 5 is a bar graph depicting the effect of the fucoxanthin composition of the invention (440 μg/mL) on lipid accumulation inhibition, under a fatty liver model of Oleate:Palmitate (O:P; 0.3 mM) in human hepatocytes (Hep G2), compared to control (“Veh”) and to 10 μM of commercial fucoxanthin either alone or in combinations with different Docosahexaenoic acid (DHA) (20, 10 and 5 μM). * p<0.0001 vs. Vehicle control without O:P; #p<0.0001 vs. Vehicle control with O:P; and $ p<0.0001 vs. Vehicle control with O:P.

FIG. 6 is a bar graph depicting the effect of the fucoxanthin composition of the invention (440 μg/mL) on lipid accumulation inhibition, under a fatty liver model of Oleate:Palmitate (O:P; 0.3 mM) in human hepatocytes (Hep G2), compared to control (“Veh”), and to 10 μM of commercial fucoxanthin either alone or in combinations with different Eicosapentaenoic acid (EPA) (20, 10, 5 and 2.5 μM). EPA alone was also examined (20 μM). * p<0.0001 vs. Vehicle control without O:P; and #p<0.0001 vs. Vehicle control with O:P.

FIG. 7 is a bar graph depicting the effect of the fucoxanthin composition of the invention (440 μg/mL) on lipid accumulation inhibition, under a fatty liver model of Oleate:Palmitate (O:P; 0.3 mM) in human hepatocytes (Hep G2), compared to control (“Veh”) and to 10 μM of commercial fucoxanthin in combinations with different Docosahexaenoic acid (DHA) and Eicosapentaenoic acid (EPA) (both at either 20 μM, 10 μM or 5 μM). * p<0.0001 vs. Vehicle control without O:P; #p<0.0001 vs. Vehicle control with O:P; and $ p<0.0009 vs. Vehicle control with 0:P.

FIG. 8 is a bar graph depicting the effect of the fucoxanthin composition of the invention (440 μg/mL) on lipid accumulation inhibition, under a fatty liver model of Oleate:Palmitate (O:P; 0.3 mM) in human hepatocytes (Hep G2), compared to control (“Veh”), and to Lutein at various concentrations (presented as μM and μg/mL). * p<0.0001 vs. Vehicle control without O:P; #p<0.0001 vs. Vehicle control with O:P; and $ p<0.0003 vs. Vehicle control with O:P.

FIG. 9 is a bar graph depicting the effect of the fucoxanthin composition of the invention (440 μg/mL) on lipid accumulation inhibition, under a fatty liver model of Oleate:Palmitate (O:P; 0.3 mM) in human hepatocytes (Hep G2), compared to control (“Veh”), and to Silymarin at various concentrations (presented as μM and μg/mL). * p<0.0001 vs. Vehicle control without O:P; and #p<0.0001 vs. Vehicle control with O:P; and $ p<0.0003 vs. Vehicle control with O:P.

FIG. 10 is a bar graph depicting the effects of serially reduced concentrations of the fucoxanthin composition of the invention solubilized in medium-chain triglyceride (MCT) oil on lipid accumulation inhibition, under a fatty liver model of Oleate:Palmitate (O:P; 0.3 mM) in human hepatocytes (Hep G2) compared to control (“Veh”) (presented as μM and μg/mL). * p<0.05 vs. Veh treated group without O:P; #p<0.05 vs. Veh treated group with O:P.

FIG. 11 is a bar graph depicting the effects of serially reduced concentrations of the fucoxanthin composition of the invention solubilized in coconut oil on lipid accumulation inhibition, under a fatty liver model of Oleate:Palmitate (O:P; 0.3 mM) in human hepatocytes (Hep G2) compared to control (“Veh”) (presented as μM and μg/mL). * p<0.05 vs. Veh treated group without O:P; #p<0.05 vs. Veh treated group with O:P.

FIG. 12 is a bar graph depicting the “carrier-effect” of medium-chain triglyceride (MCT) oil on lipid accumulation inhibition, under a fatty liver model of Oleate:Palmitate (O:P; 0.3 mM) in human hepatocytes (Hep G2), compared to the fucoxanthin composition of the invention (220 μg/mL) and control (“Veh”) (presented as μM and μg/mL). * p<0.05 vs. Veh treated group without O:P; #p<0.05 vs. Veh treated group with O:P.

FIG. 13 is a bar graph depicting the “carrier-effect” of coconut oil on lipid accumulation inhibition, under a fatty liver model of Oleate:Palmitate (O:P; 0.3 mM) in human hepatocytes (Hep G2), compared to the fucoxanthin composition of the invention (220 μg/mL) and control (“Veh”) (presented as μM and μg/mL). * p<0.05 vs. Veh treated group without O:P; #p<0.05 vs. Veh treated group with O:P.

FIG. 14 is a bar graph depicting the “carrier-effect” of Sea buckthorn oil on lipid accumulation inhibition, under a fatty liver model of Oleate:Palmitate (O:P; 0.3 mM) in human hepatocytes (Hep G2), compared to three batches of fucoxanthin composition of the invention (i.e., batch 1-220 μg/mL; batch 3-220 μg/mL; and batch 5; 110, 220, and 440 μg/mL) and controls: “Veh”, and Fucoxanthin of the invention and Sea buckthorn oil (440 μg/mL each). * p<0.05 vs. Veh treated group without O:P; #p<0.05 vs. Veh treated group with O:P.

FIG. 15 is a bar graph depicting the effect of fucoxanthin composition of the invention mixed with Sea buckthorn oil on lipid accumulation inhibition, under a fatty liver model of Oleate:Palmitate (O:P; 0.3 mM) in human hepatocytes (Hep G2). Batch 5 of fucoxanthin composition of the invention (as in FIG. 14) was mixed with 15% or 5% of Sea buckthorn (For. 15% and For. 5%, respectively). * p<0.05 vs. Veh treated group without O:P; #p<0.05 vs. Veh treated group with O:P.

FIG. 16 is a bar graph depicting the effect of various concentrations (μg/mL) of the fucoxanthin composition of the invention on lipid accumulation inhibition, under a fat accumulation model in human kidney cells (HK-2), compared to control (“Veh”). * p<0.0001 vs. Vehicle control without O:P; and #p<0.0001 vs. Vehicle control with O:P.

### DETAILED DESCRIPTION OF THE INVENTION

In some embodiments, the invention is directed to compositions comprising fucoxanthin derived from a microalgae extract, for use in prevention or treatment of a metabolic syndrome.

The present invention is based, in part, on the findings that a microalgae extract comprising fucoxanthin inhibited lipid accumulation under a fatty liver and fatty kidney models in human hepatocytes and kidney cells, respectively. Unexpectedly, a microalgae extract comprising fucoxanthin was surprisingly superior to fucoxanthin from a different source or other comparable anti-oxidants and known fatty acids.

According to some embodiments, the present invention is directed to a method of reducing lipid accumulation in a cell, the method comprising contacting the cell with therapeutically effective amount of the composition of the invention.

In some embodiments, a cell accumulating fat is designated to fat accumulation, i.e., an adipocyte. In some embodiments, the cell is an ectopic fat accumulating cell. As used herein, the term “an ectopic fat accumulating cell”, refers to any non-fat accumulating cell per se, i.e., not an adipocyte (of either white or brown adipose tissue). In some embodiments, an ectopic fat accumulating cell comprises fat, lipid droplets, or both. In some embodiments, an ectopic fat accumulating cell comprises ectopic fat, ectopic lipid droplets, or both.

In some embodiments, the ectopic fat accumulating cell is any one of: a liver cell, a kidney cell, a pancreatic cell, and a muscle cell. In some embodiments, the muscle cell is a skeletal muscle cell or a cardiac muscle cell. In some embodiments, the pancreatic cell is any one of: a β-cell, an α-cell, a γ-cell, and a δ-cell. Each possibility represents a separate embodiment of the invention.

According to some embodiments, the present invention is directed to a method of treating a metabolic syndrome in a subject in need thereof, the method comprising administering to the subject an effective amount of the composition of the invention. In one embodiment, the metabolic syndrome is a fatty liver disease.

According to another embodiment, the method comprises administering to the subject a therapeutically effective amount of a composition comprising fucoxanthin derived from microalgae and a carrier, thereby preventing or treating a metabolic syndrome in the subject.

According to some embodiments, the present invention is directed to a composition comprising fucoxanthin (5% up to 99%) and coconut oil (1% up to 95%). According to some embodiments, the composition comprises fucoxanthin and coconut oil at a ratio ranging from 100:1, 90:1, 80:1, 70:1, 60:1, 50:1, 40:1, 30:1, 20:1, 10:1, 5:1, 3:1, 1:1, 1:3, 1:5, 1:10, 1:20, 1:30, 1:40, 1:50, 1:60, 1:70, 1:80, or 1:90 to 1:100 fucoxanthin to coconut oil, respectively, or any value and range therebetween. Each possibility represents a separate embodiment of the invention.

### Treatment of Metabolic Syndrome

The term “treatment” and its cognates refer to all actions that can improve or beneficially change the symptoms of a disease, including slowing the progression, or reducing the severity of a disease and associated conditions or symptoms. The term treatment does not require a complete cure of a disease.

The term “prevention” and its cognates refer to refers to all actions that can inhibit or delay the onset or development of a disease.

As used herein the term “metabolic syndrome” refers to any disease, disorder, or condition characterized by any one of the following: excess abdominal fat, hypertension, abnormal fasting plasma glucose level or insulin resistance, high triglyceride levels, and low high-density lipoprotein (HDL) cholesterol level. The metabolic syndrome which can be treated according to the present invention include but are not limited to fatty liver, obesity, pre-diabetes, diabetes, hyperglycemia, diabetic dyslipidemia, hyperlipidemia, hypertriglyceridemia, hypercholesterolemia, hyperinsulinemia, insulin-resistance or insulin-resistance related.

In one embodiment, the metabolic syndrome is a fatty liver disease. The term “fatty liver disease” (FLD) refers to a disease or a pathological condition caused by, at least in part, abnormal hepatic lipid deposits. Fatty liver disease includes, e.g., alcoholic fatty liver disease, nonalcoholic fatty liver disease, and acute fatty liver of pregnancy. Fatty liver disease may be, e.g., macrovesicular steatosis or microvesicular steatosis. In one embodiment, the metabolic syndrome is nonalcoholic steatohepatitis (i.e., fatty liver). In one embodiment, the compositions and methods of the invention are useful for treating and preventing hepatotoxicity, nephrotoxicity or both, wherein the hepatotoxicity, nephrotoxicity, or both are fat-induced. As used herein, fat induced-hepatotoxicity, and fat-induced nephrotoxicity, refer to any disease or condition encompassing increased toxicity to cells of the liver, kidney, or both which is attributed to the accumulation of fat, triglycerides, free fatty acid, lipid droplet, or any equivalent thereof, which negatively impacts the intoxicated cell. Intoxicated cells, for example, undergo necrosis, lose membrane integrity, undergo cell lysis, cease growing or cell division, actively induce apoptosis.

In one embodiment, the metabolic syndrome is a fatty kidney disease (FKD). The term “fatty kidney disease” encompasses any disease or a pathological condition comprising ectopic lipid deposits in the kidney.

In some embodiments, the metabolic syndrome is diabetes. In some embodiments, diabetes is type 2 diabetes (T2DM). In some embodiments, the metabolic syndrome is an insulin resistance-related disease. In some embodiments, the metabolic syndrome is an insulin resistance-related-T2DM. In some embodiments, the metabolic syndrome is an insulin resistance-dependent-T2DM. In some embodiments, the metabolic syndrome is a muscular disease. In some embodiments, the metabolic syndrome is a metabolic muscular disease. In some embodiments, the metabolic syndrome is a cardiac muscle metabolic disease (i.e., cardiometabolic disease). In some embodiments, the metabolic syndrome is a skeletal muscle metabolic disease.

In some embodiments, a subject in need of treatment by the compositions of the invention may be one who is at increased risk of developing FLD, FKD, or both. For example, a subject having abnormal fat metabolism, alcoholism, advanced age (e.g., greater than 40, 50, 60, or 70 years of age), celiac disease, diabetes mellitus (e.g., type II diabetes mellitus), dyslipidemia, exposure to industrial solvents, galactosemia, glycogen storage diseases, homocystinuria, hyperferritinemia, hyperinsulinemia, hyperlipidemia, hypertension, hypertriglyceridemia, hyperuricemia, hypoxia, impaired fasting glycemia, inborn metabolic disorders (e.g., related to galactose, glycogen, homocysteine, or tyrosine metabolism), insulin resistance, iron overload, jejunal bypass surgery, low levels of high-density lipoprotein, Madelung's lipomatosis, malnutrition, Mauriac syndrome, metabolic syndrome, mitochondrial dysfunction, mitochondrial injury, mitochondrialopathies, niacin deficiency, Niemann-Pick disease, obesity (especially visceral adiposity or central obesity), overnutrition, pantothenic acid deficiency, peroxisomal diseases, polycystic ovarian syndrome, pregnancy, rapid weight loss, riboflavin deficiency, sleep apnea, starvation, tyrosemia, Weber-Christian disease, or Wilson's disease may have, or be at increased risk of developing, a disorder associated with hepatic lipid deposits. NAFLD has also been associated with rapid weight loss. In addition, patients treated with certain medications, such as, e.g., amiodarone, corticosteroids, estrogens (e.g., synthetic estrogens), maleate, methotrexate, perhexyline, salicylate, tamoxifen, tetracycline, and valproic acid may have, or be at increased risk of developing, a disorder associated with hepatic lipid deposits.

In some embodiments, a subject in need of treatment of FLD can be diagnosed by multiple methods. As would be apparent to one of ordinary skill in the art, such methods include, but are not limited to, physical examination, blood test of liver enzymes, imaging and tissue biopsy. In one embodiment, physical examination for detecting fatty liver in a subject includes seeking for an enlarged liver. In another embodiment, physical examination for detecting fatty liver in a subject includes examination of the subject's medical history of alcohol, medication, or supplement use. In one embodiment, blood test for detecting fatty liver in a subject includes measuring the concentrations of liver enzymes, such as, but not limited to, aspartate transaminase (AST) and alanine transaminase (ALT). In another embodiment, concentration of liver enzymes may be represented by the calculated ratio of AST to ALT, as apparent to one skilled in the art. In one embodiment, imaging methods for detecting fatty liver in a subject include, but not limited to, ultrasound, computational tomography (CT) or magnetic resonance imaging (MRI), and others. In one embodiment, detecting fatty liver in a subject includes collection of a tissue biopsy. In another embodiment, detecting fatty liver in a subject's tissue biopsy includes sectioning and staining. In another embodiment, one skilled in the art will appreciate that specific markers may be employed for the detection of specifically expressed genes instead of a general chemical stain.

### Pharmaceutical Compositions

According to one aspect, the composition of the invention comprises Phaeodactylum extract. In some embodiments, the composition of the invention comprises Phaeodactylum oleoresin. Pharmaceutical compositions for use in the methods of the invention are provided. The compositions of the invention comprise fucoxanthin, such as from microalgae extract, and a pharmaceutically acceptable carrier (excipient). Examples of suitable pharmaceutical carriers are described in, e.g., Martin, 1990, Remington's Pharmaceutical Sciences, 17^{th} ed. (Mack Pub. Co., Easton, Pa.). Suitable excipients may include coconut oil starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The compositions of the invention may also contain pH buffering reagents and wetting or emulsifying agents. The compositions may further contain other active compounds providing supplemental, additional, or enhanced therapeutic functions. The pharmaceutical compositions may also be included in a container, pack, or dispenser together with instructions for administration.

Suitable pharmaceutically acceptable salts of the compounds of the invention may also be included in the pharmaceutical compositions. Examples of salts include salts of inorganic acids (such as, e.g., hydrochloric, hydrobromic, phosphoric, metaphosphoric, nitric, and sulfuric acids) and of organic acids (such as, e.g., acetic, benzenesulfonic, benzoic, citric, ethanesulfonic, fumaric, gluconic, glycolic, isethionic, lactic, lactobionic, maleic, malic, methanesulfonic, succinic, p-toluenesulfonic, and tartaric acids). Other suitable pharmaceutically acceptable basic salts include ammonium salts, alkali metal salts (such as, e.g., sodium and potassium salts) and alkaline earth metal salts (such as, e.g., magnesium and calcium salts). Furthermore, the compounds of the invention may be present as a hydrate or hemihydrate (of the compound or of its salt).

The compositions can be formulated in solid (e.g., powder, tablets), liquid (e.g., aqueous or nonaqueous solutions, dispersions, suspensions or emulsions) or other forms.

Examples of suitable aqueous and nonaqueous carriers, diluents, solvents or vehicles include water (e.g., pyrogen-free water), ethanol, polyols (such as glycerol, propylene glycol, polyethylene glycol, and the like), and suitable mixtures thereof, vegetable oils (such as olive oil, cotton seed oil, palm oil, etc.), and injectable organic esters such as ethyl oleate. Proper fluidity can be maintained, for example, by the use of coating materials such as lecithin, by the maintenance of the required particle size in the case of dispersions, and by the use of surfactants. These compositions may also contain adjuvants, antibacterial and antifungal agents, flavoring agents, biodegradable polymers, etc. A non-limiting example for a plant-derived oil includes a vegetable oil.

The pharmaceutical compositions of this invention can be administered to mammals (e.g., humans, rodents, etc.) in any suitable way including, e.g., orally, parenterally, intracisternally, intraperitoneally, topically, etc. The parenteral administration includes intravenous, intramuscular, intraperitoneal, intrasternal, subcutaneous and intraarticular injection/infusion.

The dose will vary depending on the subject and upon the particular route of administration used. Commercially available assays may be employed to determine optimal dose ranges and/or schedules for administration. Effective doses may be extrapolated from dose-response curves obtained from animal models. In general, suitable animal models include (1) genetic models such as, e.g., the ob/ob mouse, fa/fa (Zucker) rat, or db/db mouse; (2) overnutrition models, in which animals are fed, e.g., a high sucrose/fructose diet or a high fat diet; (3) the methionine-choline diet deficiency model, which develops steatosis and in, some to strains, fibrosis; and (4) transgenic models, such as mice that overexpress the transcription factor SREBP-1 that governs lipid synthesis.

In some embodiments, composition of the present invention is consumed daily. In one embodiment, a daily dose of the composition comprises at least 0.1 mg, at least 0.2 mg, at least 0.5 mg, at least 0.7 mg, at least 1 mg, at least 2 mg, at least 3 mg, at least 4 mg, at least 5 mg, at least 6 mg, at least 7 mg, at least 8 mg, at least 9 mg, at least 10 mg, at least 11 mg, at least 12 mg, at least 13 mg, at least 14 mg, at least 15 mg, at least 16 mg, at least 17 mg, at least 18 mg, at least 19 mg, or at least 20 mg of fucoxanthin (e.g., within the microalgae extract), or any value and range therebetween. Each possibility represents a separate embodiment of the invention. In one embodiment, a daily dose of the composition comprises 0.1-0.2 mg, 0.15-0.5 mg, 0.35-0.7 mg, 0.6-1 mg, 0.8-2 mg, 1.5-3 mg, 2-4 mg, 2.5-5 mg, 4-6 mg, 5-7 mg, 5.5-8 mg, 6-9 mg, 7.5-10 mg, 8-11 mg, 9-12 mg, 10-13 mg, 11-14 mg, 12-15 mg, 12.5-16 mg, 14-17 mg, 13-18 mg, 16.5-19 mg or 17-20 mg of fucoxanthin. Each possibility represents a separate embodiment of the invention.

### Microalgae Extract

According to some embodiments, the composition of the invention comprises microalgae contents, e.g., fucoxanthin. According to some embodiments, the fucoxanthin of the invention is derived or extracted from microalgae.

As used herein, the term “microalgae” means any unicellular, photosynthetic microorganism, whether wild type or genetically modified microalgae. In one embodiment, the microalgae extract is extracted from diatom microalgae. In one embodiment, the microalgae extract is extracted from Phaeodactylum tricornutum. In one embodiment, the microalgae extract is extracted from Navicula pelliculosa. In one embodiment, the microalgae extract is extracted from Amphora. In one embodiment, the microalgae extract is extracted from Isochrysis aff. Galbana. In one embodiment, the microalgae extract is extracted from Odontella aurita. In one embodiment, the microalgae extract is extracted from Nitzscia closterium. In one embodiment, the microalgae extract is extracted from Cylindrotheca closterium. In one embodiment, the microalgae extract is extracted from Chaetoseros sp. In one embodiment, the microalgae extract is extracted from Emiliania huxleyi.

In one embodiment, a composition comprising a carrier such as coconut oil, may comprise fucoxanthin extracted or derived from a macro-algae. As used herein, the term “macro-algae” refers to any of macroscopic or multicellular algae. In one embodiment, the macro-algae is a wild type macro-algae, or a genetically modified macro-algae.

According to embodiments wherein the compositions comprise a carrier such as coconut oil, the fucoxanthin may be produced synthetically. As defined herein, “produced synthetically” refers to a substance which was not extracted or obtained from its natural source. Non-limiting examples may include chemical synthesis or recovery from genetically modified sources.

In one embodiment, the microalgae extract comprises at least 0.001%, at least 0.1%, at least 1%, at least 2%, at least 3% fucoxanthin by dry weight. In one embodiment, the microalgae extract comprises fucoxanthin in an amount of 1-15% by dry weight. In one embodiment, the microalgae extract comprises fucoxanthin in an amount of 15-90%

In one embodiment, the microalgae extract comprises fucoxanthin and one or more carotenoids selected from the group consisting of: Violaxanthin, Diadinoxanthin, Antheraxanthin, Diatoxanthin, Zeaxanthin, β,β-Carotene, and β,ε-Carotene.

In one embodiment, the microalgae extract further comprises fatty acids. In one embodiment, the fatty acids constitute more than 40%, or alternatively more than 45%, or alternatively more than 50% by dry weight of the microalgae extract, or any value and range therebetween. Each possibility represents a separate embodiment of the invention. In one embodiment, the fatty acids constitute 35-45%, or alternatively 40-55%, or alternatively 50-95% by dry weight of the microalgae extract. Each possibility represents a separate embodiment of the invention. In one embodiment, the fatty acids are selected from the group consisting of: saturated fatty acids, unsaturated fatty acids, trans fatty acids and any combinations thereof.

In one embodiment, the fatty acids are selected from the group consisting of: saturated fatty acids, unsaturated fatty acids, trans fatty acids and any combinations thereof.

In one embodiment, the fatty acids are selected from the group consisting of: saturated fatty acids, mono-unsaturated fatty acids, poly-unsaturated fatty acids, trans fatty acids or any combinations thereof. In one embodiment, the saturated fatty acids are one or more fatty acids selected from the group consisting of: arachidic acid, arachidonic acid, butyric acid, caproic acid, capric acid, caprylic acid, lauric acid, myristic acid, pentadecanoic acids, palmitic acid (PA), palmitoleic acid, heptadecenoic acid, stearic acid, behenic acid, lignoceric acid, or isomers thereof. In one embodiment, the mono-unsaturated fatty acids are one or more fatty acids selected from the group consisting of: myristoleic acid, palmitoleic acid, pentadecanoic acid, oleic acid, heptadecanoic acid, vaccenic acid, docosenic acid, or isomers thereof. In one embodiment, the to poly-unsaturated fatty acids are one or more fatty acids selected from the group consisting of: eicosapentaenic acid (EPA), linoleic acid, alpha linolenic acid, gamma linolenic acid, docosapentaenic acid, docosahexaenoic acid (DHA), or isomers thereof. In one embodiment, the trans fatty acid are one or more fatty acids selected from the group consisting of: trans-oleic acid, trans-vaccenic acid and trans isomers w/o trans OI/Vac.

In one embodiment, the microalgae extract further comprises palmitoleic acid or isomers thereof, wherein the palmitoleic acid constitutes more than 0.1%, or alternatively more than 0.5%, or alternatively more than 1%, or alternatively more than 2%, or alternatively more than 3%, or alternatively more than 4%, or alternatively more than 5%, or alternatively more than 6%, or alternatively more than 7%, or alternatively more than 8%, or alternatively more than 9%, or alternatively more than 10%, or alternatively more than 11%, or alternatively more than 12%, or alternatively more than 13%, or alternatively more than 14% by dry weight of the microalgae extract, or any value and range therebetween. Each possibility represents a separate embodiment of the present invention.

In one embodiment, the microalgae extract further comprises arachidonic acid (AA) or isomers thereof, wherein the AA constitute more than 0.1% or alternatively more than 0.2%, or alternatively more than 0.5%, or alternatively more than 0.6%, or alternatively more than 0.7%, or alternatively more than 0.9%, or alternatively more than 1%, or alternatively more than 1.5%, or alternatively more than 2%, or alternatively more than 2.5%, or alternatively more than 3% by dry weight of the microalgae extract, or any value and range therebetween. Each possibility represents a separate embodiment of the present invention.

In one embodiment, the microalgae extract further comprises docosahexaenoic acid (DHA) or isomers thereof, wherein the DHA constitute more than 0.01%, or alternatively more than 0.05%, or alternatively more than 0.1%, or alternatively more than 0.2% by dry weight of the microalgae extract, or any value and range therebetween. Each possibility represents a separate embodiment of the present invention.

In one embodiment, the microalgae extract further comprises palmitic acid (PA) or isomers thereof, wherein the PA constitute more than 5%, or alternatively more than 7%, or alternatively more than 7%, or alternatively more than 9%, or alternatively more than 11%, or alternatively more than 15% by dry weight of the microalgae extract, or any value and range therebetween. Each possibility represents a separate embodiment of the present invention.

In one embodiment, the microalgae extract comprises fucoxanthin and fatty acids. In some embodiments, the weight to weight ratio of the fucoxanthin to the fatty acids in the extract ranges between 1:10 and 1:30. In some embodiments, the weight to weight ratio of the fucoxanthin to the fatty acids, ranges between 1:10 and 1:20.

In some embodiments, the extract comprises fucoxanthin and unsaturated fatty acids. In some embodiments, the weight to weight ratio of the fucoxanthin to the unsaturated fatty acids in the extract ranges between 1:2 and 1:3, 1:3 and 1:5, 1:5 and 1:10 or 1:3 and 1:15. In some embodiments, the unsaturated fatty acids comprise monounsaturated fatty acids and polyunsaturated fatty acids.

In some embodiments, the weight to weight ratio of the fucoxanthin to the mono and poly unsaturated fatty acids in the extract ranges between 1:1 and 1:3, 1:2 and 1:4, 1:3 and 1:5 or 1:4 and 1:10. In some embodiments, the weight to weight ratio of the fucoxanthin to the poly-unsaturated fatty acids of the extract ranges between 1:1 and 1:30, 1:2 and 1:20, 1:3 to 1:15, 1:3 to 1:10, 1:4 and 1:30, 1:4 and 1:20, 1:4 to 1:15, 1:4 to 1:10, 1:5 and 1:30, 1:5 and 1:20, 1:5 to 1:15 or 1:5 and 1:10. In some embodiments, the weight to weight ratio of the fucoxanthin to the mono-unsaturated fatty acids of the extract ranges between 1:1 and 1:10, 1:2 and 1:20, 1:3 to 1:15, 1:3 to 1:10, 1:4 and 1:30, 1:4 and 1:20, 1:4 to 1:15, 1:4 to 1:10, 1:5 and 1:30, 1:5 and 1:20, 1:5 to 1:15 or 1:5 and 1:10.

In one embodiment, the microalgae extract comprises fucoxanthin, palmitoleic acid, eicosapentaenic acid (EPA), arachidonic acid (AA), gamma linolenic acid, docosahexaenic acid (DHA) and palmitic acid (PA) or isomers thereof.

In one embodiment, the palmitoleic acid and/or isomers thereof constitute more than 0.1%, or alternatively more than 0.5%, or alternatively more than 1%, or alternatively more than 2%, or alternatively more than 3%, or alternatively more than 4%, or alternatively more than 5%, or alternatively more than 6%, or alternatively more than 7%, or alternatively more than 8%, or alternatively more than 9%, or alternatively more than 10%, or alternatively more than 11%, or alternatively more than 12%, or alternatively more than 13%, or alternatively more than 14% by dry weight of the microalgae extract, or any value and range therebetween. Each possibility represents a separate embodiment of the present invention.

In one embodiment, the microalgae extract comprises fucoxanthin and palmitoleic acid or isomers thereof. In some embodiments, the weight to weight ratio of the fucoxanthin to the palmitoleic acid in the extract ranges between 30:1 and 1:10, 20:1 and 1:8, 2:1 and 1:2, 1:1 and 1:10, 3:1 and 1:5, 1:1 and 1:2, 1:2 and 1:10, or 1:2 and 1:5. Each possibility represents a separate embodiment of the present invention.

In one embodiment, the eicosapentaenic acid (EPA) or isomers thereof constitute more than 0.075%, or alternatively more than 1.5%, or alternatively more than 2%, or alternatively more than 3%, or alternatively more than 4%, or alternatively more than 5%, or alternatively more than 6%, or alternatively more than 7%, or alternatively more than 8%, or alternatively more than 10%, or alternatively more than 11%, or alternatively more than 12%, or alternatively more than 13%, or alternatively more than 14%, or alternatively more than 15%, or alternatively more than 16%, or alternatively more than 17%, or alternatively more than 18%, or alternatively more than 19%, or alternatively more than 20%, or alternatively more than 21%, or alternatively more than 22%, or alternatively more than 23%, or alternatively more than 24%, or alternatively more than 25% by dry weight of the microalgae extract, or any value and range therebetween. Each possibility represents a separate embodiment of the present invention.

In one embodiment, the microalgae extract comprises fucoxanthin and EPA. In some embodiments, the weight to weight ratio of the fucoxanthin to the EPA in the extract ranges between 25:1 and 1:25, 20:1 and 1:20, 10:1 and 1:10, 1:1 and 1:10, 5:1 and 1:5, 1:1 and 1:2, 1:2 and 1:10, 1:2 and 1:8, 1:2 and 1:7, or 1:2 and 1:6. Each possibility represents a separate embodiment of the present invention. In some embodiments, the weight to weight ratio of the fucoxanthin to the EPA in the extract ranges between 4:1 and 1:8.

In some embodiments, the extract comprises fucoxanthin and arachidonic acid (AA) or isomers thereof. In some embodiments, the weight to weight ratio of the fucoxanthin to the AA in the extract ranges between 10:1 and 1:2, 15:1 and 1:2, 20:1 and 1:2, 1:1 and 1:2, 1.5:1 and 1:1.5, 4:1 and 1:1, 3:1 and 1:1, 2:1 and 1:1, or 1.5:1 and 1:1. Each possibility represents a separate embodiment of the present invention. In some embodiments, the weight to weight ratio of the fucoxanthin to the AA in the extract ranges between 15:1 and 1:1. In some embodiments, the weight to weight ratio of the fucoxanthin to the AA in the extract ranges between 10:1 and 1:1.

In one embodiment, DHA or isomers thereof constitute more than 0.05%, or alternatively more than 0.075%, or alternatively more than 0.1%, or alternatively more than 0.15%, or alternatively more than 0.2% by dry weight of the microalgae extract, or any value and range therebetween. Each possibility represents a separate embodiment of the present invention.

In one embodiment, the microalgae extract comprises fucoxanthin and DHA. In some embodiments, the weight to weight ratio of the fucoxanthin to the DHA in the extract ranges between 30:1 and 1:1, 20:1 and 1:1, 10:1 and 1:1, 7:1 and 1:1, 6:1 and 1:1, 4:1 and 1:1, 10:1 and 2:1, 8:1 and 2:1, 7:1 and 2:1, 6:1 and 2:1, 5:1 and 2:1, 4:1 and 2:1, 10:1 and 3:1, 8:1 and 3:1, 7:1 and 3:1, 6:1 and 3:1, 5:1 and 3:1, or 4:1 and 3:1. Each possibility represents a separate embodiment of the present invention. In some embodiments, the weight to weight ratio of the fucoxanthin to the DHA in the extract ranges between 6:1 and 2:1. In some embodiments, the weight to weight ratio of the fucoxanthin to the DHA in the extract ranges between 5:1 and 3:1.

In one embodiment, the PA or isomers thereof constitute more than 5%, or alternatively more than 6%, or alternatively more than 7%, or alternatively more than 8%, or alternatively more than 8.5% by dry weight of the microalgae extract, or any value and range therebetween. Each possibility represents a separate embodiment of the present invention.

In one embodiment, the microalgae extract comprises fucoxanthin and PA. In some embodiments, the weight to weight ratio of the fucoxanthin to the PA in the extract ranges between 2:1 and 1:10, 2:1 and 1:8, 2:1 and 1:7, 2:1 and 1:6, 2:1 and 1:5, 2:1 and 1:4, 1:1 and 1:10, 1:1 and 1:8, 1:1 and 1:7, 1:1 and 1:6, 1:1 and 1:5, 1:1 and 1:4, 1:2 and 1:10, 1:2 and 1:8, 1:2 and 1:7, 1:2 and 1:6, 1:2 and 1:5, 1:2 and 1:4, 1:3 and 1:10, 1:3 and 1:8, 1:3 and 1:7, 1:3 and 1:6, or 1:3 and 1:5. Each possibility represents a separate embodiment of the present invention.

In some embodiments, the weight to weight ratio of the fucoxanthin to the PA in the extract ranges between 1:3 and 1:5. In some embodiments, the weight to weight ratio of the fucoxanthin to the PA in the extract ranges between 1:2 and 1:6.

The microalgae may be grown in a culture medium known to one skilled in the art. A suitable culture medium is any medium known in the art that support the viability and growth of the microalgae.

As used herein, the microalgae extract refers to materials extracted from microalgae. In one embodiment, microalgae can be harvested prior to extraction by any conventional means including, but not limited to filtration, air flotation and centrifugation.

The extraction of fucoxanthin and additional ingredients may be carried out by any means known in the art. In one embodiment, the extraction is a mechanical extraction. In another embodiment, the extraction is carried out by using an organic solvent. In one embodiment, the organic solvent is at least partially miscible in water. Non-limiting example of solvents that are miscible in water include methanol, ethanol, propanol, isopropanol, n-propanol, other alcohols containing 4 carbons or less, acetone, ketones containing 4 carbons or less, cyclic ethers such as dioxane and tetrahydrofuran, water miscible ethers such as diethyl ether, other oxygen-containing organic molecules having a ratio of carbon to oxygen atoms of about 4:1 or less and acetonitrile, or combination thereof. In another embodiment, the organic solvent is immiscible in water. Non-limiting examples of organic solvent that are immiscible in water include alkanes such as hexane, pentane, heptane, octane, esters such as ethyl acetate, butyl acetate, ketones such as methyl ethyl ketone (MEK), methyl isobutyl ketone (MIBK), aromatics such as toluene, benzene, cyclohexane, tetrahydrofuran, haloalkanes such as chloroform, trichloroethylene and ethers such as diethyl ether, or combinations thereof.

The term “polar solvent” as used herein means a solvent that tends to interact with other compounds or itself through acid-base interactions, hydrogen bonding, dipole-dipole interactions, or by dipole-induced dipole interactions. Non-limiting examples of polar solvents include: ethanol, propylene glycol, butylene glycol, methanol, glycerol, propanol, butanol, dipropylene glycol, pentylene glycol, hexylene glycol, dimethyl formamide, acetonitrile, dimethyl sulfoxide, dichloromethane, ethyl acetate, tetrahydrofuran, formic acid, acetic acid and acetone. Each possibility represents a separate embodiment of the invention. According to yet additional embodiments, the extraction is performed with a combination of at least two solvents.

In another embodiment, the extraction is carried out by using supercritical fluid-CO₂ (SCF-CO₂) as known in the art. As used herein, supercritical fluid-CO₂ refer to CO₂ at a temperature (e.g., 40-60° C.) and pressure above its critical point, where distinct liquid and gas phases do not exist. In one embodiment, supercritical fluid-CO₂ can effuse through solids like a gas, and dissolve materials like a liquid. In another embodiment, the extraction is carried out by using SCF-CO₂ and a co-solvent. In one embodiment, the co-solvent is selected from ethanol, acetone, methanol, and any combination thereof.

In one embodiment, an extraction by a solvent is carried out following the SCF—CO₂ extraction. In one embodiment, the extraction with a solvent is a liquid-liquid extraction. In one embodiment, the solvent is a polar solvent. In one embodiment, the solvent is selected from the group consisting of: ethanol, methanol, acetone, hexane and heptane. In some embodiment, the extraction by a solvent is followed by a second extraction by a second solvent. In some embodiments, the second solvent is a polar solvent.

The term “liquid-liquid extraction”, also known as solvent extraction and partitioning, refers to an extraction of a substance from one liquid into another liquid phase. In liquid-liquid extraction, substances are separated based on their relative solubilities in two different immiscible liquids (solvents), such as for a non-limiting example water and an organic solvent.

For a non-limiting example, the extraction is carried out by using supercritical fluid-CO₂ (SCF—CO2), followed by an extraction by a polar solvent, such as ethanol to enrich the ethanol extracted mass, which is followed by a second extraction with a second polar solvent (e.g., ethanol, ketone, ester, etc.).

In some embodiments, microalgae extract is further fractionated. As defined herein, “fractionation” refers to any process by which multiple substances of a mixture are divided and subsequently collected into a number of smaller quantities (i.e., fractions) each comprised of several components sharing a specific property. Methods of fractionation are apparent to one skilled in the art, and non-limiting examples include adsorption, capillary electrophoresis, centrifugation, cyclonic separation, chromatography, crystallization, decantation, demister, distillation, drying, electrophoresis, electrostatic separation, elutriation, evaporation, extraction, field flow fractionation, flotation, flocculation, filtration, fractional filtration, fractional freezing, oil-water separation, magnetic separation, precipitation, recrystallization, scrubbing, sedimentation, sieving, stripping, sublimation, vapor-liquid separation, winnowing and zone refining, and others.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the to present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds.), “Basic and Clinical Immunology” (8^{th} Edition), Appleton & Lange, Norwalk, Conn. (1994); Mishell and Shiigi (eds.), “Strategies for Protein Purification and Characterization—A Laboratory Course Manual” CSHL Press (1996); all of which are incorporated by reference. Other general references are provided throughout this document.

### Materials and Methods

### Microalgae Growth and Cultivation

Phaeodactylum microalgae were cultured at 20° C., under constant aeration supplemented with 2% CO₂, and pH of 7.5±0.5, in artificial seawater medium (JONES, R. F., H. L. SPEER, AND W. KURY. 1963). The culture was harvested upon reaching a minimum biomass of 3.5 gr/L.

TABLE 1
The biomass and oleoresin contents (determined under two separate
experiments) compared to macro-algae oleoresin content:

| | | | | Macro- |
|---|---|---|---|---|
| | P. tricornutum | | | algae |
| | Dry biomass | Oleoresin 1 | Oleoresin 2 | Oleoresin |
| | [%] | [%] | [%] | [%] |
| Fucoxanthin | 1.5-2 | 3.12 | 3.12 | 5.52 |
| Protein | 40.90 | 0.74 | 0.11 | NA |
| Total fat | 11.53 | 95.89 | 85 | 91.4 |
| Caprylic acid | <0.02 | 7.62 | 5.35 | 48.17 |
| in product | | | | |
| Capric acid | 0.01 | 5.39 | 3.9 | 42.32 |
| in product | | | | |
| Palmitic acid | 3.26 | 7.8 | 6.14 | <0.1 |
| in product | | | | |
| Arachidonic acid | 0.35 | 0.21 | 0.17 | <0.1 |
| in product | | | | |
| EPA in product | 3.53 | 0.82 | 2.04 | <0.1 |
| DHA in product | 0.21 | 0.09 | 0.11 | <0.1 |
| Total UFA | 9.47 | 10.38 | 11.9 | 0.55 |
| in product | | | | |
| Total PUFA | 4.77 | 2.92 | 4.66 | 0.37 |
| in product | | | | |
| Total MUFA | 4.24 | 6.95 | 7.27 | 0.18 |
| in product | | | | |
| Total saturated | 3.34 | 81.22 | 73.01 | 90.85 |
| FA in product | | | | |
| Glucose | 2.63 | <0.1 | <0.1 | NA |
| Sum of mono and | 2.63 | <0.7 | <0.7 | NA |
| disaccharides | | | | |
| Sodium | 1.73 | 0.02 | 0.25 | NA |

### In Vitro Fatty Liver Model in Human Hepatocytes (Hep G2)

Hep G2 cells were incubated overnight in 96-well plates (4×10⁴ cells/well) with Roswell Park Memorial Institute medium (RPMI) supplemented with 10% fetal bovine serum (FBS). Then cells were exposed to a mixture of fatty acids (0.3 mM Oleate:Palmitate, 2:1) in the presence of a vehicle (“Veh”) or tested compounds for an additional 24 hours. Following the incubation period, the cells were washed, incubated with 1 μg/mL mixture of Nile-Red/Hoechst solution for 15 min at 37° C. protected from light. Fluorescence was measured by the Cytation 3-plate reader applying excitation at 530 nm and recording emission at 635 nm for Nile-Red and to applying excitation at 350 nm and recording emission at 461 nm for Hoechst. Results were normalized to total protein levels of each well and presented as a change in the accumulation of lipids in comparison with vehicle-treated group. Data represented mean±SEM from 8 replicates per group.

### In Vitro Fatty Kidney Model in Human Kidney Cells (HK-2)

Human kidney HK-2 cells were maintained at 37° C. in 5% CO₂ in DMEM medium supplemented with 10% FCS, 2 mM L-glutamine, 1 mM sodium pyruvate, 100 U/mL penicillin, and 100 mg/mL streptomycin. Free fatty acids (2:1, molar ratio, oleic and palmitic acids) were mixed with bovine serum albumin (BSA). Cells were incubated with fatty acid-BSA complex in FCS-free culture medium at 1 mmol/L final concentration of fatty acid and 1% of BSA. Control cell cultures were incubated with medium containing the vehicle. Compounds were tested in a range of concentrations in the presence/absence of fatty acids. After 24 hr of incubation with the compounds, the cells were washed, incubated with 1 μg/mL mixture of Nile-Red/Hoechst solution for 15 mins at 37° C. protected from light. Fluorescence was measured by the Cytation-3 plate reader at ex: 530 nm/em: 635 nm and ex: 350 nm/em: 461 nm for Nile-Red and Hoechst, respectively. Results were normalized to Hoechst reading in each well, and presented as a change in the accumulation of lipids in comparison with Vehicle-treated group.

### Example 1

### The Effect of Fucoxanthin Derived from Phaeodactylum Extract on Lipid Accumulation in Human Hepatocytes (Hep G2 Cells)

Initially, the inventors examined the ability of the Phaeodactylum extract to reduce intracellular lipid accumulation. A dosage-dependent negative correlation was observed (FIG. 1), and Phaeodactylum extract concentration of 440 μg/mL which comprises 13.2 μg/mL of fucoxanthin was determined as a ‘set-point’ for proceeding experiments.

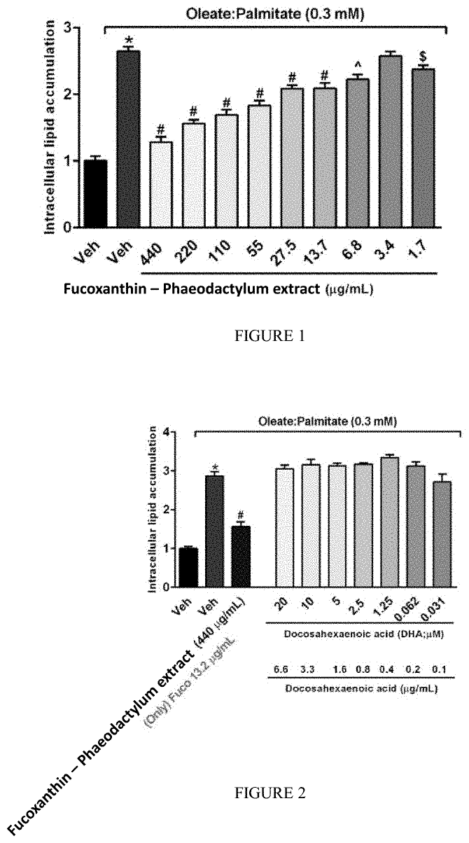

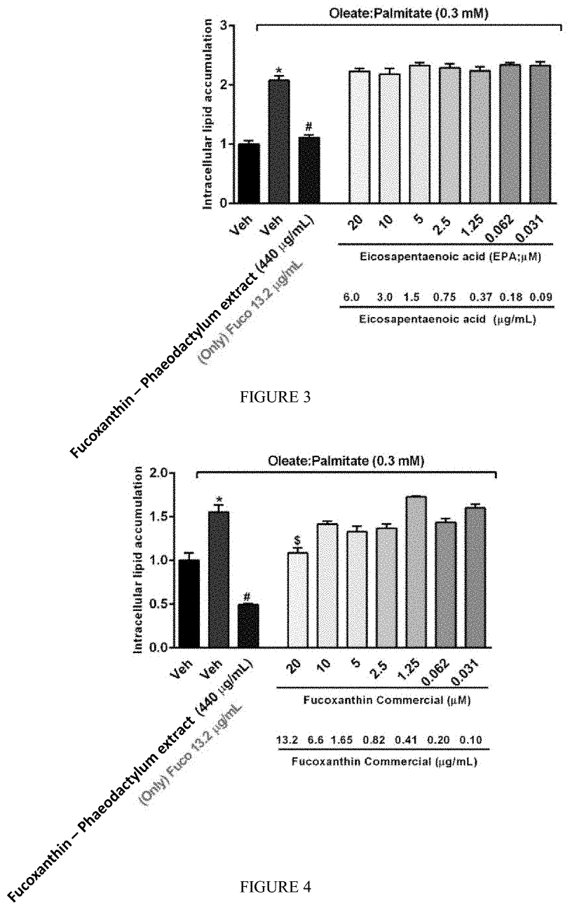

Then, the inventors examined the potency of the above Phaeodactylum extract set point in comparison to DHA and EPA. In both cases (e.g., DHA and EPA) the set-point of the Phaeodactylum extract was found to be significantly more potent in reducing lipid accumulation (FIGS. 2 and 3, respectively). Furthermore, the inventors have demonstrated that the Phaeodactylum extract set point was more efficient than purified fucoxanthin from seaweed with to respect to lipid accumulation inhibition, even when the same amounts of purified seaweed's fucoxanthin and fucoxanthin of Phaeodactylum extract were supplied to Hep G2 cells (FIG. 4).

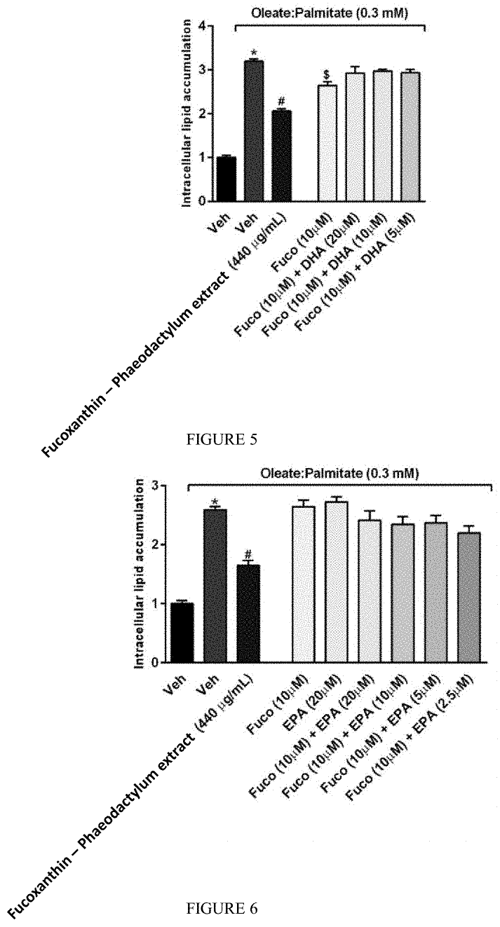

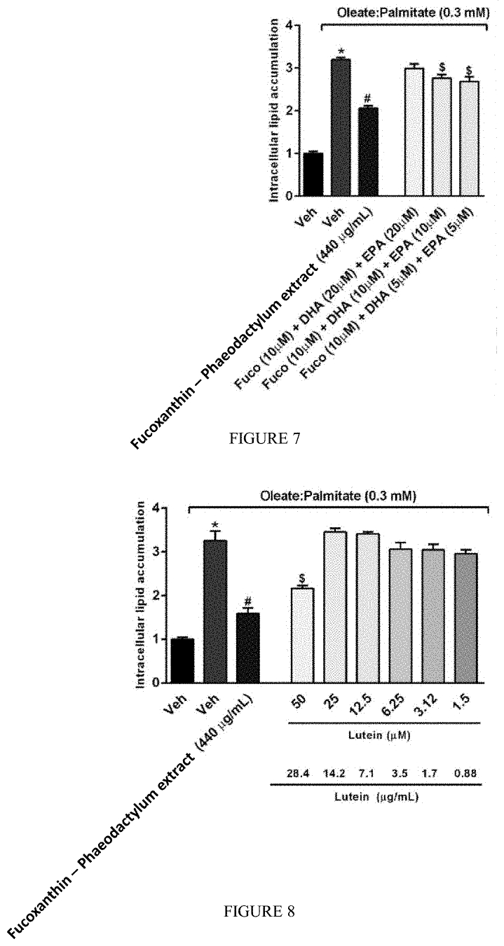

Subsequently, the Phaeodactylum extract set point was examined in comparison to purified fucoxanthin of seaweed in combinations with either DHA (FIG. 5), EPA (FIG. 6), and DHA and EPA (FIG. 7). The Phaeodactylum extract set point was found to be significantly more effective in reducing intracellular lipid accumulation than any of the three combinations.

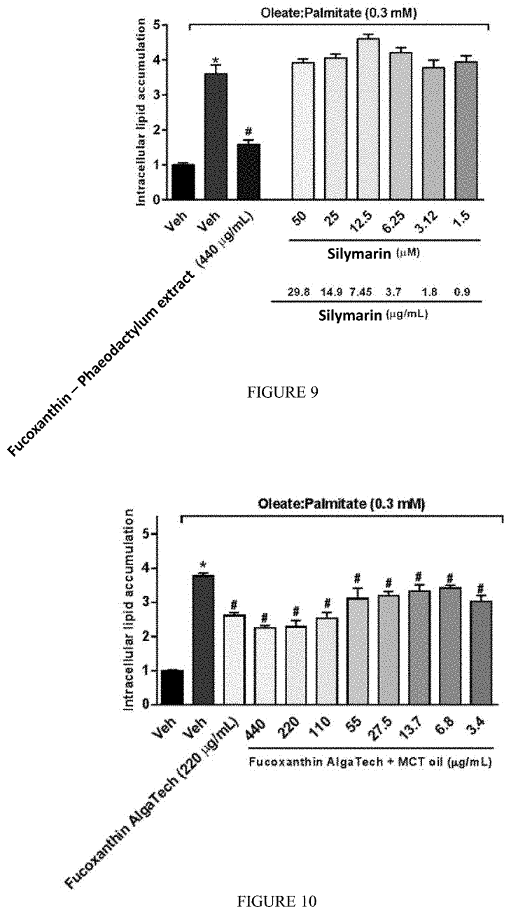

To further test the efficacy of the Phaeodactylum extract, the current Phaeodactylum extract composition was compared with commercial antioxidants susceptible of having hepatoprotective activity. Accordingly, the inventors compared the Phaeodactylum extract set point to Lutein and Silymarin in the fatty liver model of human hepatocytes in vitro (FIGS. 8 and 9, respectively). In both cases, the Phaeodactylum extract was shown to be significantly more potent in reducing lipid accumulation in the Hep G2 cells.

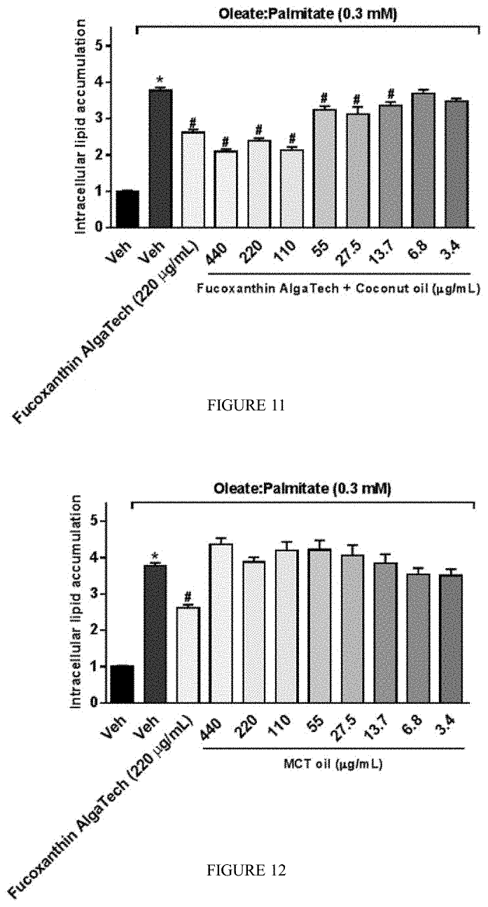

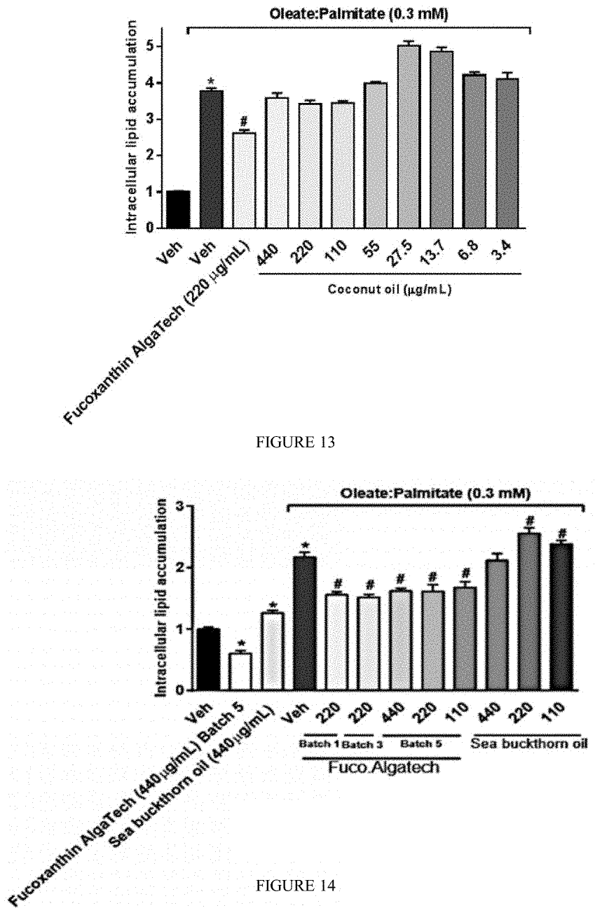

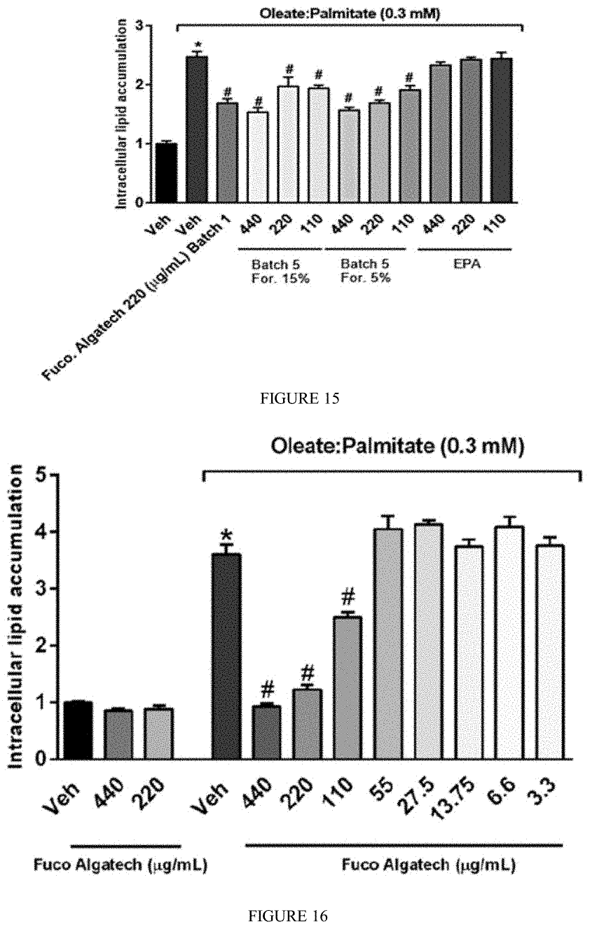

To further test whether the beneficial effect of reduced lipid accumulation was attributed solely to the Phaeodactylum extract and not partially contributed by the carrier (e.g., coconut oil medium-chain triglyceride (MCT), or sea buckthorn oil), hepatoprotective activity of the extract was compared to that of a carrier. Accordingly, the inventors compared the Phaeodactylum extract set point in the presence of a carrier (in dose dependency manner) to a carrier-only, in the fatty liver model of human hepatocytes in vitro (FIGS. 10-11 and 12-13, respectively), or compared different batches of Phaeodactylum extracts in the presence of a carrier (in dose dependency manner; FIGS. 14-15). In all cases, the Phaeodactylum extract was shown to be the sole contributor to the reduction of lipid accumulation in the Hep G2 cells. No significant difference was observed with respect to the efficacy of Phaeodactylum extract solubilized with either coconut oil or MCT. Nonetheless, sea buckthorn oil only may have had even induced fat accumulation at some doses.

All in all, various batches of microalgae cultures comprising fucoxanthin were used in the aforementioned examples, all of which were proved to be advantageous over the controls with respect to reducing lipid accumulation in human hepatocytes (FIGS. 1-15).

### Example 2

### The Effect of Fucoxanthin Derived from Phaeodactylum Extract on Lipid Accumulation in Human Kidney Cells (HK-2 Cells)

The Phaeodactylum extract was shown to be highly effective in reducing lipid accumulation in the HK-2 kidney cells, in a dose dependent matter (FIG. 16).

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for reducing lipid content in a cell, the method comprising the step of contacting said cell with a composition comprising microalgae extract and a carrier, thereby decreasing lipid accumulation in said cell, thereby reducing the lipid content in the cell.

2. The method of claim 1, wherein said cell is an ectopic fat accumulating cell.

3. The method of claim 1, wherein said ectopic fat accumulating cell is selected from the group consisting of: a liver cell, a kidney cell, a pancreatic cell, and a muscle cell.

4. A method of preventing or treating a metabolic syndrome in a subject in need thereof, the method comprising administering to the subject a therapeutically effective amount of a composition comprising microalgae extract and a carrier, thereby preventing or treating a metabolic syndrome in the subject.

5. The method of claim 4, wherein said metabolic syndrome is selected from the group consisting of: a fatty liver disease, and a fatty kidney disease.

6. The method of claim 1, wherein said microalgae extract is a Phaeodactylum extract.

7. The method of claim 6, wherein said Phaeodactylum extract comprises at least 0.5% (w/w) fucoxanthin by dry weight.

8. The method of claim 6, wherein said Phaeodactylum is Phaeodactylum tricornutum.

9. The method of claim 1, wherein said carrier is a plant-derived oil.

10. The method of claim 9, wherein said plant-derived oil comprises at least 85% fatty acid chains comprising 16 carbon atoms at most.

11. The method of claim 10, wherein said plant-derived oil comprises at least 85% saturated fatty acid chains.

12. The method of claim 11, wherein said plant-derived oil is coconut oil or medium-chain triglyceride (MCT) oil.

13. The method of claim 1, wherein said microalgae extract is a fraction of a microalgae extract.

14. The method of claim 13, wherein said fraction comprises at least 0.03 μg/mL fucoxanthin.

15. The method of claim 1, wherein said fraction comprises one or more compounds selected from the group consisting of: palmitic acid, arachidonic acid, eicosapentaenic acid, and docosahexaenoic acid.

16. The method of claim 1 for preventing or treating fat-induced hepatotoxicity, nephrotoxicity, or both.
